(19) United States
(12) Patent Application Publication
(10) Pub. No.: US 2023/0036368 A1
(43) Pub. Date: Feb. 2, 2023

(54) INFORMATION PROCESSING DEVICE, INFORMATION PROCESSING METHOD, AND INFORMATION PROCESSING PROGRAM

(71) Applicant: FUJIFILM CORPORATION, Minato-ku, Tokyo (JP)

(72) Inventors: Yasuhisa KANEKO, Kanagawa (JP); Yasuyuki HOSONO, Kanagawa (JP); Nobuya KITAMURA, Kanagawa (JP); Kenji NAGAMIYA, Kanagawa (JP); Tomohide HIRAGAMI, Kanagawa (JP)

(21) Appl. No.: 17/812,726

(22) Filed: Jul. 15, 2022

(30) Foreign Application Priority Data
Jul. 29, 2021 (JP) 2021-124691

(51) Int. Cl. A61B 5/00

(52) U.S. Cl. CPC A61B 5/0022; A61B 5/0026; A61B 5/7275

(57) ABSTRACT

An information processing device comprising at least one processor, wherein the at least one processor is configured to: receive information transmitted to a user; and issue, to a measurement device that measures biological information of the user, a command regarding the measurement in a case where the received information includes risk information, which is information that may affect health or safety of the user, regarding a situation around the user.

### CROSS-REFERENCE TO RELATED APPLICATIONS

The present application claims priority under 35 U.S.C. § 119 to Japanese Patent Application No. 2021-124691, filed on Jul. 29, 2021. The above application is hereby expressly incorporated by reference, in its entirety, into the present application.

### BACKGROUND

### Technical Field

The present disclosure relates to an information processing device, an information processing method, and an information processing program.

### Related Art

In the related art, a technique has been known in which biological information is monitored according to a health condition of a user. For example, JP2019-117612A discloses that a measurement time and interval of a biological information measurement device are decided based on user's health degree information classified by a diagnosis result by a doctor, a blood pressure measurement result, a user's self-report, and the like.

In recent years, there has been a demand for a technique that contributes to early detection of a disease by monitoring biological information such as a body temperature and a heart rate. In particular, since some people infected with various infectious diseases such as a coronavirus disease have no subjective symptoms, the early detection of the infection by monitoring the biological information is considered to be very effective in preventing the spread of infection.

On the other hand, continuing to measure the biological information in a case of absence of events that affect the user's health may not be preferable since the continuous measurement is bothersome for the user or power consumption of a biological information measurement device increases. Thus, there is a demand for a technique that enables appropriate measurement of the biological information as necessary.

### SUMMARY

The present disclosure provides an information processing device, an information processing method, and an information processing program capable of appropriately checking a health condition of a user.

An information processing device according to a first aspect of the present disclosure comprises at least one processor. The processor receives information transmitted to a user and issues, to a measurement device that measures biological information of the user, a command regarding the measurement in a case where the received information includes risk information, which is information that may affect health or safety of the user, regarding a situation around the user.

In the first aspect, the processor may decide a measurement condition of the biological information by the measurement device based on the risk information and issue the command to the measurement device according to the decided measurement condition.

In the first aspect, the risk information may include information indicating a probability of infection of the user with an infectious disease.

In the first aspect, the risk information may include information indicating a health condition of another person around the user.

In the first aspect, the risk information may include information indicating an infection state of another person around the user with an infectious disease.

In the first aspect, the risk information may include information about a disaster or pollution around the user.

In the first aspect, the risk information may further include a proposal for the measurement, and the processor may issue the command regarding the measurement to the measurement device according to the proposal included in the risk information.

In the first aspect, the command may be to start the measurement.

In the first aspect, the command may be to transmit a result of the measurement to the information processing device.

In the first aspect, the command may be to change a measurement condition in the measurement.

In the first aspect, the processor may receive the risk information via at least one of e-mail or social networking service (SNS).

An information processing method according to a second aspect of the present disclosure executed by a computer comprises receiving information transmitted to a user and issuing, to a measurement device that measures biological information of the user, a command regarding the measurement in a case where the received information includes risk information, which is information that may affect health or safety of the user, regarding a situation around the user.

An information processing program according to a third aspect of the present disclosure causing a computer to execute a process comprises receiving information transmitted to a user and issuing, to a measurement device that measures biological information of the user, a command regarding the measurement in a case where the received information includes risk information, which is information that may affect health or safety of the user, regarding a situation around the user.

According to the above aspect, the information processing device, the information processing method, and the information processing program of the present disclosure can appropriately check the health condition of the user.

### BRIEF DESCRIPTION OF THE DRAWINGS

FIG. 1 is a schematic configuration diagram of an information processing system.

FIG. 2 is a block diagram showing an example of a hardware configuration of an information processing device.

FIG. 3 is a block diagram showing an example of a functional configuration of the information processing device.

FIG. 4 is a table showing an example of risk information and measurement conditions.

FIG. 5 is a diagram showing an example of a screen displayed on a display.

FIG. 6 is a flowchart showing an example of first information processing.

FIG. 7 is a diagram showing an example of a screen displayed on the display.

FIG. 8 is a flowchart showing an example of second information processing.

### DETAILED DESCRIPTION

Hereinafter, embodiments for implementing a technique of the present disclosure will be described in detail with reference to drawings.

### First Embodiment

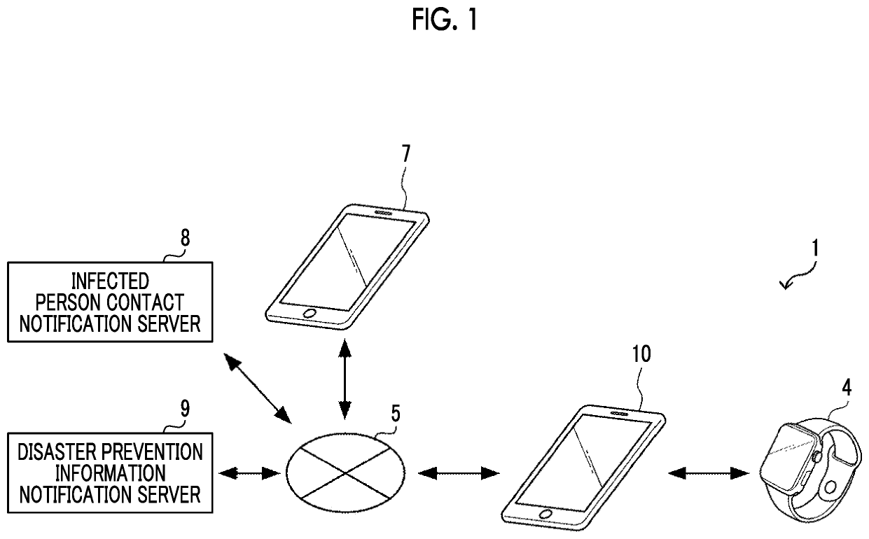

First, a configuration of an information processing system 1 according to the present embodiment will be described with reference to FIG. 1. As shown in FIG. 1, the information processing system 1 comprises an information processing device 10 and a measurement device 4. FIG. 1 illustrates a smartphone as an example of the information processing device 10. The information processing device 10 and the measurement device 4 can communicate with each other by wired or wireless communication. Further, the information processing device 10 is connected, in a communicable state, to external devices such as a smartphone 7 owned by another person, an infected person contact notification server 8, and a disaster prevention information notification server 9 via a wired or wireless network 5.

The measurement device 4 has a function of measuring at least one type of biological information of a user. The biological information indicates, for example, at least one of body temperature, heartbeat, electrocardiogram, myoelectricity, blood pressure, arterial oxygen saturation (SpO2), blood glucose level, lipid level, or the like. In these cases, for example, a wearable terminal such as a smartwatch provided with a thermometer, a heart rate monitor, a blood glucose self-measurement device, a sensor that measures the biological information such as heartbeat and arterial oxygen saturation, and the like can be applied as the measurement device 4. In a case where there is a command to measure the biological information, the measurement device 4 may measure the biological information only once or may measure the biological information over time. The term “measurement over time” means to continuously measure the biological information at preset time intervals.

Further, the measurement device 4 may have a function of transmitting the measured biological information to the information processing device 10 by wired or wireless communication. As the wireless communication standard in this case, for example, Wi-Fi (registered trademark), Bluetooth (registered trademark), and the like can be applied as appropriate. Although FIG. 1 shows one measurement device 4, the number of measurement devices 4 included in the information processing system 1 according to the present embodiment is not particularly limited. For example, in a case where the information processing system 1 includes a plurality of measurement devices 4, each of the plurality of measurement devices 4 may be enabled to communicate with the information processing device 10 by wired or wireless communication. Further, each of the plurality of measurement devices 4 may measure different types of the biological information.

The infected person contact notification server 8 uses a known coronavirus contact check application to have a function of notifying, in a case where the user who is an owner of the information processing device 10 may have contacted a person infected with a coronavirus disease, the information processing device 10 of the fact. The disaster prevention information notification server 9 has a function of notifying, in a case where a disaster, such as fire and gas leak, or pollution has occurred in an area where the user who is the owner of the information processing device 10 stays, the information processing device 10 of the fact. The infected person contact notification server 8 and the disaster prevention information notification server 9 are, for example, servers in which a software program that provides a server function is installed in a general-purpose computer.

The information processing device 10 according to the present embodiment issues, to the measurement device 4, the command regarding the measurement of the biological information of the user according to various pieces of information transmitted from the external devices such as the smartphone 7 owned by another person, the infected person contact notification server 8, and the disaster prevention information notification server 9. Hereinafter, a detailed configuration of the information processing device 10 will be described.

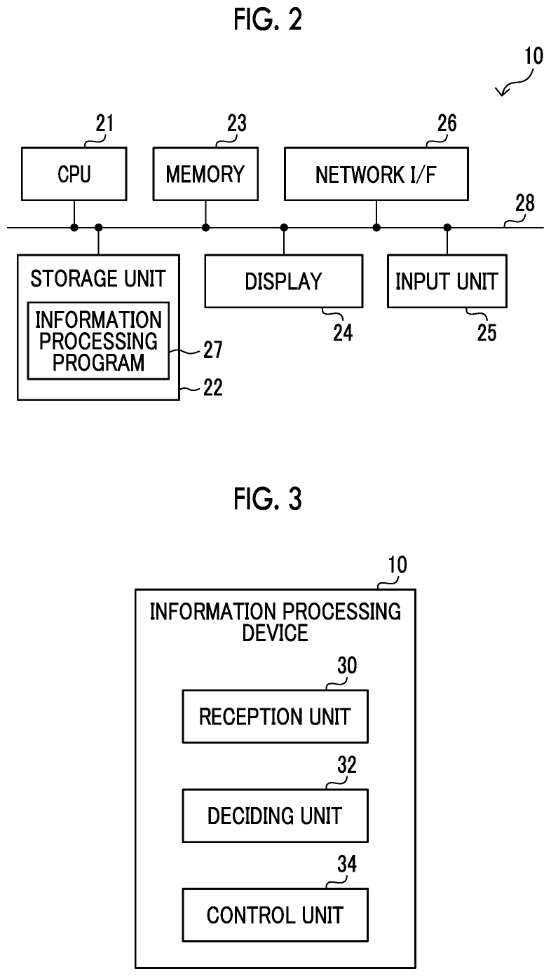

Next, an example of a hardware configuration of the information processing device 10 according to the present embodiment will be described with reference to FIG. 2. As shown in FIG. 2, the information processing device 10 includes a central processing unit (CPU) 21, a nonvolatile storage unit 22, and a memory 23 as a temporary storage area. The information processing device 10 further includes a display 24 such as a liquid crystal display, an input unit 25 such as a touch panel and a button, and a network interface (I/F) 26. The network I/F 26 performs wired or wireless communication with the measurement device 4 and the network 5. The CPU 21, the storage unit 22, the memory 23, the display 24, the input unit 25, and the network I/F 26 are mutually connected, via a bus 28 such as a system bus and a control bus, such that various pieces of information can be exchanged.

The storage unit 22 is formed by a recording medium such as a hard disk drive (HDD), a solid state drive (SSD), and a flash memory. The storage unit 22 stores an information processing program 27 in the information processing device 10. The CPU 21 reads out the information processing program 27 from the storage unit 22, develops the readout program into the memory 23, and executes the developed information processing program 27. The CPU 21 is an example of a processor of the present disclosure. As the information processing device 10, for example, a personal computer, a tablet terminal, a wearable terminal, and the like can be applied as appropriate, in addition to the smartphone illustrated in FIG. 1.

Next, an example of a functional configuration of the information processing device 10 according to the present embodiment will be described with reference to FIG. 3. As shown in FIG. 3, the information processing device 10 includes a reception unit 30, a deciding unit 32, and a control unit 34. The CPU 21 executes the information processing program 27 to function the CPU 21 as the reception unit 30, the deciding unit 32, and the control unit 34.

The reception unit 30 receives, by the network I/F 26, information transmitted to the user via the network 5 from the external devices such as the smartphone 7 owned by another person, the infected person contact notification server 8, and the disaster prevention information notification server 9. The “information transmitted to the user” may be, for example, a text-format message transmitted from the smartphone 7 owned by another person to the information processing device 10. In this case, the reception unit 30 may receive the information transmitted to the user via at least one of e-mail or social networking service (SNS). The e-mail is not limited to e-mail using a protocol such as simple mail transfer protocol (SMTP) and includes, for example, various services having a function of transmitting and receiving messages such as webmail and short message service (SMS). The SNS includes various services having a function of transmitting and receiving messages such as Twitter (registered trademark), Facebook (registered trademark), and LINE (registered trademark).

The “information transmitted to the user” may be, for example, a notification, which is transmitted from the infected person contact notification server 8, indicating that the user may have contacted the person infected with the coronavirus disease. The “information transmitted to the user” may be, for example, a notification, which is transmitted from the disaster prevention information notification server 9, indicating that the disaster, such as fire and gas leak, or pollution has occurred in the area where the user stays.

The “information transmitted to the user” is not limited to the information transmitted to an individual user, but may be information transmitted to a group to which the user belongs. For example, the term “information transmitted to the user” may be information transmitted using a group talk, a mailing list, or the like known in various services having a function of transmitting and receiving messages. For example, the term “information transmitted to the user” may be information transmitted by a service that provides various pieces of information about disasters, pollution, or an epidemic situation of an infectious disease to a plurality of terminal devices in a corresponding area, using position information of a terminal device (including the information processing device 10).

The deciding unit 32 determines whether or not the information received by the reception unit 30 includes risk information that may affect the health or safety of the user. Specifically, the “risk information” is at least one of information that may affect the health or safety of the user and about a situation around the user or information about a health condition of the user.

For example, the “risk information” may be information indicating a probability of infection of the user with the infectious disease. An example of such information includes the notification, which is transmitted from the infected person contact notification server 8, indicating that the user may have contacted the person infected with the coronavirus disease. An example of such information further includes keywords indicating the probability of infection of the user with the infectious disease such as “influenza”, “COVID-19”, “food poisoning”, “infection”, “infect” and “contact”, which are included in a message received by the reception unit 30.

For example, the “risk information” may be information indicating the health condition of another person around the user. The “risk information” may be information indicating an infection state of another person around the user with the infectious disease, among the information indicating the health condition of another person around the user. An example of such information includes keywords indicating the health condition of another person around the user such as “fever”, “went to hospital”, and “infected with influenza”, which are included in the message received by the reception unit 30.

For example, the “risk information” may be information about disasters or pollution around the user. An example of such information includes the notification, which is transmitted from the disaster prevention information notification server 9, indicating that the disaster, such as fire and gas leak, or pollution has occurred in the area where the user stays.

For example, the “risk information” may be the information indicating the health condition of the user. Examples of such information include a diagnosis result by a doctor and a result of a health examination. These pieces of information may be transmitted from, for example, a server computer (not shown) that manages an electronic medical record.

In a case where the deciding unit 32 determines that the information received by the reception unit 30 includes the risk information, the control unit 34 issues, to the measurement device 4, the command regarding the measurement of the biological information of the user. In a case where the risk information as described above is received, the health condition of the user may change. Therefore, it is preferable to check the health condition of the user by monitoring the biological information with the measurement device 4.

For example, the “command” may be a command to start the measurement of the biological information by the measurement device 4. Specifically, the “command” may be a command to measure the biological information once by the measurement device 4 or may be a command to start the measurement of the biological information over time by the measurement device 4.

For example, the “command” may be a command to transmit the measurement result of the biological information measured by the measurement device 4 to the information processing device 10. In this case, the control unit 34 may perform control to display, on the display 24, the measurement result of the biological information received from the measurement device 4.

For example, the “command” may be a command to change a measurement condition in the measurement of the biological information by the measurement device 4. The “measurement condition” may be, for example, a type of the biological information to be measured and a period of continuing the measurement, a measurement frequency, and the like in a case where the measurement device 4 measures the biological information over time. Specifically, the “measurement condition” may be, for example, a command to change a measurement frequency setting such that the measurement device 4 which is set to measure a body temperature once a day measures the body temperature three times a day.

The deciding unit 32 may decide the measurement condition of the biological information by the measurement device 4 based on the risk information. In this case, the control unit 34 issues, to the measurement device 4, the command regarding the measurement of the biological information according to the measurement condition decided by the deciding unit 32. That is, the information processing device 10 according to the present embodiment may change contents of the command to the measurement device 4 according to the risk information. With such a configuration, it is possible to measure appropriate biological information according to the risk information and thus check the health condition of the user more appropriately.

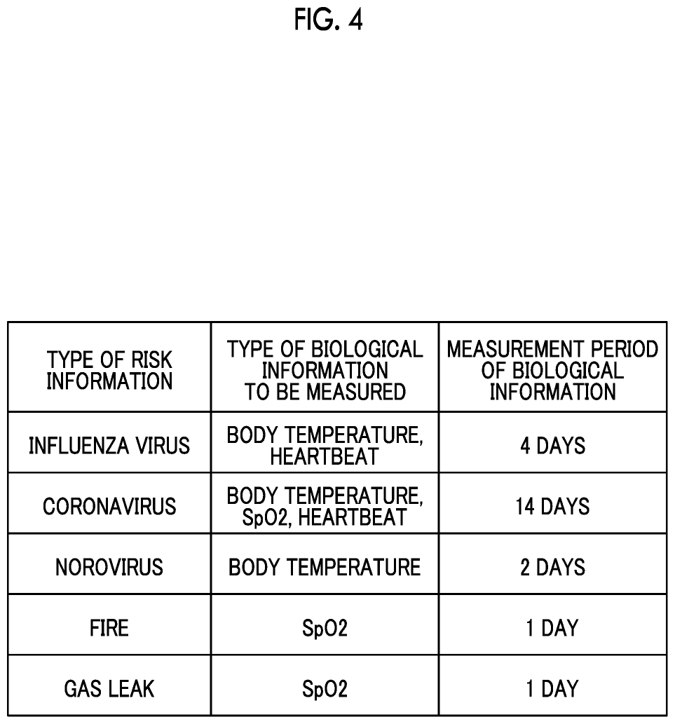

FIG. 4 shows an example of a table in which measurement conditions of the biological information are preset for each type of the risk information. For example, the deciding unit 32 may estimate the type of the risk information according to the keywords used for determining the risk information and collate the estimated type of the risk information with the table shown in FIG. 4 to decide the measurement conditions of the biological information. The estimation of the type of the risk information may be performed, for example, by the deciding unit 32 referring to a table (not shown) in which keywords and types of the risk information are associated in advance. The table in which the types of the risk information and the measurement conditions are associated in advance (refer to FIG. 4) and the table in which the keywords and the types of the risk information are associated in advance (not shown) are stored in the storage unit 22 in advance, for example.

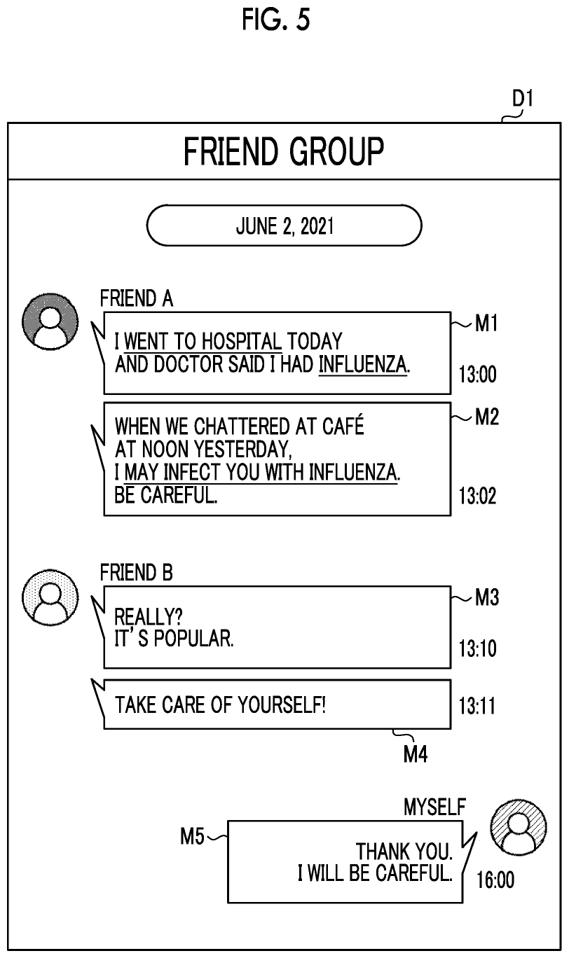

A specific example of the processing of the reception unit 30, the deciding unit 32, and the control unit 34 will be described with reference to FIGS. 4 and 5. FIG. 5 is an example of a screen D1 displayed on the display 24 of the information processing device 10. The screen D1 shows messages M1 to M4 addressed to the user received by the reception unit 30 from the smartphones 7 owned by other persons (“friend A” and “friend B”). The messages M1 and M2 are messages received from “friend A”, and the messages M3 and M4 are messages received from “friend B”. A message M5 is a message transmitted from the user oneself to “friend A” and “friend B”. Further, “Jun. 2, 2021” in FIG. 5 means that the messages M1 to M5 are exchanged on the date, and a transmission point in time of each message is described in a lower right of the messages M1 to M5.

The deciding unit 32 analyzes the messages M1 to M4 (refer to FIG. 5) received by the reception unit 30 to determine whether or not the risk information is included. As shown in the messages M1 and M2 of FIG. 5, in a case where keywords that may affect the user's health such as “went to hospital”, “influenza”, and “infect” are included, the deciding unit 32 determines that the messages M1 and M2 include the risk information.

The deciding unit 32 estimates that the risk information about an influenza virus is included based on the keyword “influenza” included in the message M1 of FIG. 5. Thereafter, the deciding unit 32 decides, by referring to the table of FIG. 4, such that the type of the biological information is set to “body temperature” and “heartbeat” and a measurement period of the biological information is set to “4 days” as the measurement conditions corresponding to the risk information about “influenza virus”.

The control unit 34 issues, to the measurement device 4, the command to start the measurement of the biological information such that the measurement device 4 measures the biological information according to the measurement conditions decided by the deciding unit 32. That is, the measurement device 4 is commanded to measure each of “body temperature” and “heartbeat” for “4 days”.

As shown in the messages M3 and M4 of FIG. 5, in a case where only a message that does not include the keyword that may affect the health or safety of the user is received, the deciding unit 32 determines that the information received by the reception unit 30 does not include the risk information. In this case, the control unit 34 does not issue, to the measurement device 4, the command regarding the measurement of the biological information.

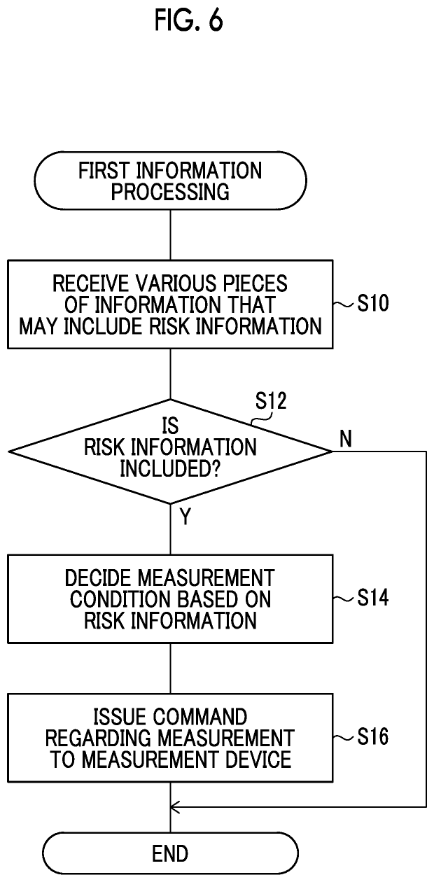

Next, an action of the information processing device 10 according to the present embodiment will be described with reference to FIG. 6. In the information processing device 10, the CPU 21 executes the information processing program 27 to execute first information processing shown in FIG. 6. The first information processing is executed, for example, in a case where the user gives an instruction to start execution via the input unit 25.

In step S10, the reception unit 30 receives various pieces of information transmitted to the user from the external devices such as the smartphone 7 owned by another person, the infected person contact notification server 8, and the disaster prevention information notification server 9. The various pieces of information received by the reception unit 30 in step S10 may include the risk information that may affect the health or safety of the user. In step S12, the deciding unit 32 determines whether or not the various pieces of information received by the reception unit 30 in step S10 include the risk information.

In a case where the various pieces of information received by the reception unit 30 in step S10 include the risk information (that is, in a case of affirmative determination in step S12), the processing proceeds to step S14, and the deciding unit 32 decides the measurement conditions of the biological information by the measurement device 4 based on the risk information. In step S16, the control unit 34 issues, to the measurement device 4, the command regarding the measurement of the biological information according to the measurement conditions decided by the deciding unit 32 in step S14. In a case where step S16 is completed, the CPU 21 ends the first information processing.

On the other hand, in a case where the various pieces of information received by the reception unit 30 in step S10 do not include the risk information (that is, in a case of negative determination in step S12), the processing of steps S14 and S16 is not performed, and the CPU 21 ends the first information processing.

As described above, the information processing device 10 according to the preferred embodiment of the present disclosure comprises at least one processor. The processor receives the information transmitted to the user and issues the command regarding the measurement to the measurement device that measures the biological information of the user in a case where the received information includes the risk information, which is the information that may affect the health or safety of the user, regarding the situation around the user. That is, with the information processing device 10 according to the present embodiment, the biological information of the user can be measured in a case where the risk information that may affect the health or safety of the user is received. Therefore, it is possible to appropriately check the health condition of the user.

As described above, the risk information that triggers the measurement of the biological information of the user in the present embodiment may be information about the situation around the user. Therefore, it is possible to start the measurement of the biological information from a timing at which there is no diagnosis by a doctor, subjective symptoms, changes in the biological information, and the like and the user's own health condition is not yet affected and contribute to early detection of changes in the health condition.

In the first embodiment, the risk information may further include a proposal for the measurement of the biological information by the measurement device 4. In this case, the processor may issue, to the measurement device 4, the command regarding the measurement of the biological information according to the proposal included in the risk information. Specifically, the deciding unit 32 may decide the measurement conditions of the biological information by the measurement device 4 by taking into account proposals received from the external devices such as the smartphone 7 owned by another person, the infected person contact notification server 8, and the disaster prevention information notification server 9. For example, the infected person contact notification server 8 is assumed to notify that the user may have contacted the person infected with the coronavirus and transmit, to the information processing device 10, information about contents of a proposal to measure SpO2 as the biological information of the user for 21 days. In this case, the deciding unit 32 may decide the measurement conditions such that each of “body temperature” and “heartbeat” is measured for 14 days based on the table shown in FIG. 4 and “SpO2” is measured for 21 days based on the proposal from the infected person contact notification server 8.

### Second Embodiment

In the first embodiment, a form has been described in which in a case where the processor receives the risk information, the command regarding the measurement of the biological information is issued to the measurement device 4. In a second embodiment, a form will be described in which in a case where the processor receives the information transmitted to the user and the received information includes the risk information, the processor acquires the biological information of the user measured in advance by the measurement device 4 according to the risk information. Hereinafter, an example of the functional configuration of the information processing device 10 according to the present embodiment will be described, and some descriptions of the same functions and configurations as those of the first embodiment will be omitted.

The reception unit 30 receives, by the network I/F 26, information transmitted to the user via the network 5 from the external devices such as the smartphone 7 owned by another person, the infected person contact notification server 8, and the disaster prevention information notification server 9. Since the “information transmitted to the user” is the same as that of the first embodiment, the description thereof will be omitted.

The deciding unit 32 determines whether or not the information received by the reception unit 30 includes the risk information that may affect the health or safety of the user. The “risk information” in the present embodiment is the same information as in the first embodiment, but may further include date and time information indicating a date and time at which a situation that may affect the health or safety of the user occurs. For example, the infected person contact notification server 8 may notify that the user may have contacted the person infected with the coronavirus and transmit, to the information processing device 10, the date and time information indicating the date and time at which the user may have contacted the infected person. For example, the message M2 in FIG. 5 includes the keywords “noon yesterday˜infect”. In this case, the deciding unit 32 may generate, based on the message M2, the date and time information indicating “12:00 on Jun. 1, 2021 (noon yesterday)” as the date and time at which the situation that may affect the health of the user occurs.

In a case where the deciding unit 32 determines that the information received by the reception unit 30 includes the risk information, the control unit 34 acquires the biological information of the user measured in advance by the measurement device 4. An acquisition source of the biological information is not particularly limited. For example, the control unit 34 may acquire the biological information from the measurement device 4. For example, in a case where the biological information measured by the measurement device 4 is sequentially transmitted to the information processing device 10 and the biological information is stored in the storage unit 22 of the information processing device 10, the control unit 34 may perform control to acquire the biological information from the storage unit 22. For example, in a case where the biological information measured by the measurement device 4 is transmitted to an external data management server (not shown), the control unit 34 may perform control to acquire the biological information from the data management server.

Further, the deciding unit 32 may decide acquisition conditions of the biological information based on the risk information. The “acquisition condition” may be, for example, a type of the biological information to be acquired, designation of a date and time and/or period of the measurement of the biological information, the number of data of the biological information to be acquired, and the like. In this case, the control unit 34 acquires the biological information according to the acquisition conditions decided by the deciding unit 32.

Specifically, the deciding unit 32 may decide a period including at least a timing according to the risk information as the designation of the date and time and/or period of the measurement of the biological information as an example of the acquisition condition. In this case, the control unit 34 acquires the biological information measured in advance during the period including at least the timing decided by the deciding unit 32 as the acquisition condition. For example, the “timing” may be a date and time at which the reception unit 30 acquires various pieces of information including the risk information. The date and time corresponds to the date and time at which the message M1 is received (13:00 on Jun. 2, 2021), in the example of FIG. 5. For example, the “timing” may be a date and time indicated by the above date and time information. The date and time corresponds to the date and time generated by the deciding unit 32 based on the message M2 (12:00 on Jun. 1, 2021), in the example of FIG. 5.

The term “period including at least the timing” means a preset period based on the above timing (for example, 12 hours before the above timing and from after the above timing to the present). With such a configuration, even in a case where the biological information is not measured just at the timing decided by the deciding unit 32, the biological information measured at a close timing can be acquired. Therefore, it is possible to appropriately check the health condition of the user. Further, in a case where a plurality of pieces of biological information measured during the period including the above timing can be acquired, a temporal change in the biological information can be checked. Therefore, it is possible to more appropriately check the health condition of the user.

The control unit 34 may perform control to display, on the display 24, at least one of the acquired biological information, an index related to the biological information, or a comparison result between the biological information and the index. The “index related to the biological information” is, for example, a threshold value to be diagnosed as abnormality in various pieces of biological information, an average value or a median value in a group having the same age and gender as the user, and an average value or a median value derived from the biological information of the user in normal times.

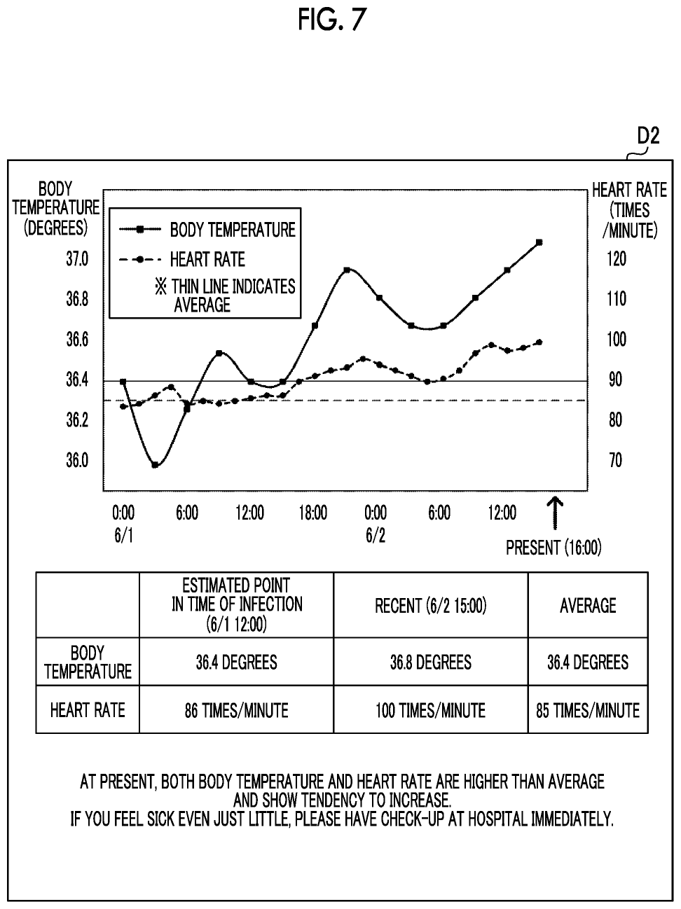

FIG. 7 shows an example of a screen D2 displayed on the display 24 by the control unit 34. On the screen D2 of FIG. 7, the body temperature and the heart rate as an example of the biological information acquired by the control unit 34 are shown as a graph. With the display of the biological information measured in advance during the period including at least the timing according to the risk information in this manner, it is possible to appropriately check the health condition of the user.

The screen D2 of FIG. 7 shows, in a field of “estimated point in time of infection”, the body temperature and the heart rate at the date and time indicated by the date and time information generated by the deciding unit 32 based on the message M2 of FIG. 5. In a field of “recent”, the body temperature and the heart rate measured recently are shown. In a field of “average”, average values of the body temperature and the heart rate derived from the biological information of the user in normal times are shown. The screen D2 of FIG. 7 illustrates a comment regarding the comparison result with the average. With the display of the acquired biological information, the index related to the biological information, and the comparison result between the biological information and the index in this manner, it is possible to check the health condition of the user in a more easily understandable manner.

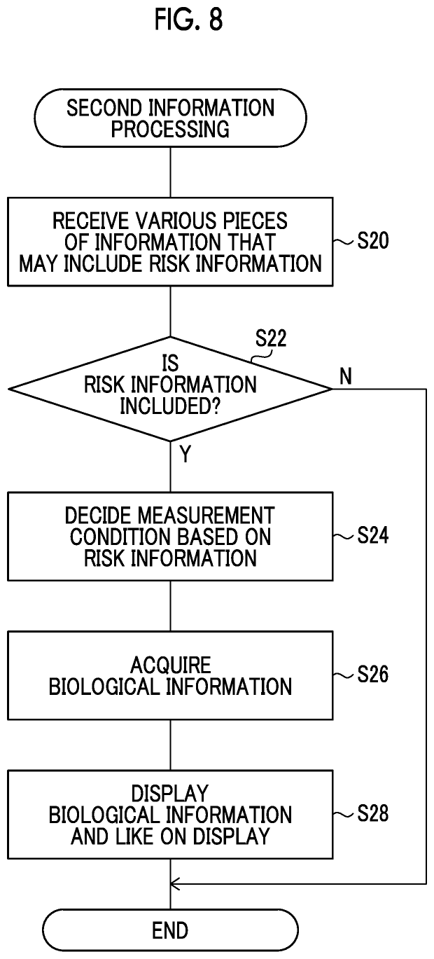

Next, an action of the information processing device 10 according to the present embodiment will be described with reference to FIG. 8. In the information processing device 10, the CPU 21 executes the information processing program 27 to execute second information processing shown in FIG. 8. The second information processing is executed, for example, in a case where the user gives the instruction to start execution via the input unit 25.

In step S20, the reception unit 30 receives various pieces of information transmitted to the user from the external devices such as the smartphone 7 owned by another person, the infected person contact notification server 8, and the disaster prevention information notification server 9. The various pieces of information received by the reception unit 30 in step S20 may include the risk information that may affect the health or safety of the user. In step S22, the deciding unit 32 determines whether or not the various pieces of information received by the reception unit 30 in step S20 include the risk information.

In a case where the various pieces of information received by the reception unit 30 in step S20 include the risk information (that is, in a case of affirmative determination in step S22), the processing proceeds to step S24, and the deciding unit 32 decides the acquisition condition of the biological information based on the risk information. In step S26, the control unit 34 acquires the biological information according to the acquisition condition decided by the deciding unit 32 in step S24. In step S28, the control unit 34 performs control to display, on the display 24, at least one of the biological information acquired in step S26, the index related to the biological information, or the comparison result between the biological information and the index. In a case where step S28 is completed, the CPU 21 ends the second information processing.

On the other hand, in a case where the various pieces of information received by the reception unit 30 in step S20 do not include the risk information (that is, in a case of negative determination in step S22), the processing of steps S24 to S28 is not performed, and the CPU 21 ends the second information processing.

As described above, the information processing device 10 according to the preferred embodiment of the present disclosure comprises at least one processor. The processor receives the information transmitted to the user and acquires the biological information of the user measured in advance according to the risk information in a case where the received information includes the risk information, which is the information that may affect the health or safety of the user. That is, with the information processing device 10 according to the present embodiment, in a case where the risk information that may affect the health or safety of the user is received, the biological information of the user measured in advance can be acquired. Therefore, it is possible to appropriately check the health condition of the user.

In the above embodiment, for example, the following various processors can be used as hardware structures of processing units that execute various pieces of processing, such as the reception unit 30, the deciding unit 32, and the control unit 34. The various processors include a programmable logic device (PLD), such as a field programmable gate array (FPGA), which is a processor whose circuit configuration is changeable after manufacturing, a dedicated electric circuit, such as an application specific integrated circuit (ASIC), which is a processor having a circuit configuration exclusively designed to execute specific processing, and the like, in addition to the CPU which is a general-purpose processor that executes software (program) to function as various processing units, as described above.

One processing unit may be configured by one of the various processors or a combination of two or more processors of the same type or different types (for example, a combination of a plurality of FPGAs or a combination of a CPU and an FPGA). The plurality of processing units may be configured of one processor.

As an example of configuring the plurality of processing units with one processor, first, there is a form in which one processor is configured by a combination of one or more CPUs and software and the processor functions as the plurality of processing units, as represented by computers such as a client and a server. Second, there is a form in which a processor that realizes the functions of the entire system including the plurality of processing units with one integrated circuit (IC) chip is used, as represented by a system-on-chip (SoC) or the like. As described above, the various processing units are configured using one or more of the various processors as the hardware structure.

Further, more specifically, a circuitry combining circuit elements such as semiconductor elements can be used as the hardware structure of the various processors.

In the above embodiment, the form in which the information processing program 27 is stored (installed) in the storage unit 22 in advance has been described, but the present disclosure is not limited thereto. The information processing program 27 may be provided in a form of being recorded on a recording medium such as a compact disc read only memory (CD-ROM), a digital versatile disc read only memory (DVD-ROM), and a Universal Serial Bus (USB) memory. The information processing program 27 may be downloaded from an external device through a network. Further, the technique of the present disclosure extends to a storage medium that stores the information processing program non-transitorily, in addition to the information processing program.

In the technique of the present disclosure, the above embodiments can be combined as appropriate. The contents described and the contents shown hereinabove are specific descriptions regarding the part according to the technique of the present disclosure and are merely an example of the technique of the present disclosure. For example, the descriptions regarding the configurations, the functions, the actions, and the effects are descriptions regarding an example of the configurations, the functions, the actions, and the effects of the part according to the technique of the present disclosure. Accordingly, in the contents described and the contents shown hereinabove, it is needless to say that removal of an unnecessary part, or addition or replacement of a new element may be employed within a range not departing from the gist of the technique of the present disclosure.

What is claimed is:

1. An information processing device comprising at least one processor, wherein the at least one processor is configured to:
receive information transmitted to a user; and
issue, to a measurement device that measures biological information of the user, a command regarding the measurement in a case where the received information includes risk information, which is information that may affect health or safety of the user, regarding a situation around the user.

2. The information processing device according to claim 1, wherein the at least one processor is configured to:
decide a measurement condition of the biological information by the measurement device based on the risk information, and
issue the command to the measurement device according to the decided measurement condition.

3. The information processing device according to claim 1, wherein the risk information includes information indicating a probability of infection of the user with an infectious disease.

4. The information processing device according to claim 1, wherein the risk information includes information indicating a health condition of another person around the user.

5. The information processing device according to claim 4, wherein the risk information includes information indicating an infection state of another person around the user with an infectious disease.

6. The information processing device according to claim 1, wherein the risk information includes information about a disaster or pollution around the user.

7. The information processing device according to claim 1, wherein:
the risk information further includes a proposal for the measurement, and
the at least one processor is configured to issue the command regarding the measurement to the measurement device according to the proposal included in the risk information.

8. The information processing device according to claim 1, wherein the command is to start the measurement.

9. The information processing device according to claim 1, wherein the command is to transmit a result of the measurement to the information processing device.

10. The information processing device according to claim 1, wherein the command is to change a measurement condition in the measurement.

11. The information processing device according to claim 1, wherein the at least one processor is configured to receive the risk information via at least one of e-mail or social networking service (SNS).

12. An information processing method executed by a computer, the method comprising:
receiving information transmitted to a user; and
issuing, to a measurement device that measures biological information of the user, a command regarding the measurement in a case where the received information includes risk information, which is information that may affect health or safety of the user, regarding a situation around the user.

13. A non-transitory computer-readable storage medium storing an information processing program causing a computer to execute a process comprising:
receiving information transmitted to a user; and
issuing, to a measurement device that measures biological information of the user, a command regarding the measurement in a case where the received information includes risk information, which is information that may affect health or safety of the user, regarding a situation around the user.
